(19) United States
(12) Patent Application Publication
(10) Pub. No.: US 2004/0198839 A1
(43) Pub. Date: Oct. 7, 2004

(54) Cardioprotective dosage units

(76) Inventors: Herbert M. Dean, Waban, MA (US); Robert E. Weinstein, Boston, MA (US); Allan M. Weinstein, Potomac, MD (US)

Correspondence Address: MESMER & DELEAULT, PLLC, 41 BROOK STREET, MANCHESTER, NH, 03104, US

(21) Appl. No.: 10/828,797

(22) Filed: Apr. 21, 2004

Related U.S. Application Data
(63) Continuation of application No. 09/717,746, filed on Nov. 21, 2000, now abandoned.
(60) Provisional application No. 60/227,249, filed on Aug. 23, 2000.

(51) Int. Cl. A61K 31/137

(52) U.S. Cl. 514/651

(57) ABSTRACT

A single dosage medication formulation incorporating a beta-adrenergic antagonist and an agent to prevent platelet aggregation to enhance simplicity, convenience, and compliance with the use of these agents.

This application is a continuation of Ser. No. 09/717,746, filed Nov. 21, 2000, which claims the benefit of U.S. Provisional Application No. 60/227,249, filed Aug. 1, 2000.

### BACKGROUND OF THE INVENTION

1. Field of the Invention

The present invention relates to treatments for reducing the risk of cardiovascular disease. Particularly, the present invention relates to combinations of agents that antagonize beta-adrenergic function and agents that reduce platelet aggregation. More particularly, the present invention relates to beta-blocker agents and agents to reduce platelet aggregation for the treatment of cardiovascular disease and for the purpose of reducing medication error and increasing therapeutic compliance.

2. Description of the Prior Art

Cardiovascular disease is responsible for about 40% of the deaths in industrialized countries. Two categories of agents are commonly utilized to reduce morbidity and mortality from these diseases: agents that reduce platelet aggregation and agents that induce blockade of the adrenergic nervous system.

Platelet aggregation is an important factor in the pathogenesis of cardiovascular diseases. Antiplatelet agents have been shown to be effective in preventing cardiovascular disease. Aspirin is an example of such an agent. Two large primary prevention trials of aspirin have been completed in healthy men. The largest of these, the Physicians' Health Study, enrolled 22,071 apparently healthy male physicians aged 40 to 84. A 44% reduction in nonfatal heart attacks was observed in those taking 325 mg of aspirin every other day. In a similar trial in Britain, an overall 32% reduction in the risk of first non-fatal heart attack appears to be associated with aspirin prophylaxis. The U.S. Preventive Services Task Force recommends aspirin for the primary prevention of myocardial infarction in men 40 years old and older in whom risk of myocardial infarction is sufficiently high to warrant risking the possible adverse effects of the drug. Meta-analysis of randomized secondary trials involving people with a history of occlusive vascular disease have demonstrated that aspirin reduces the subsequent incidence of heart attack, stroke and death by about 25% in both men and women.

Despite this compelling clinical evidence, many individuals at risk fail to benefit from such treatment. One perception underlying this failure is that the importance of aspirin in preventing cardiovascular disease may be trivialized by lay individuals because of its familiarity, its availability and its use is, therefore, dismissed. Some individuals instructed to take both a prescription medication and aspirin may assume that the prescription is more potent. Consequently, they fail to adhere to taking aspirin. Some individuals may not elect to pay for an over-the-counter product, desiring rather, to obtain a prescription that would be reimbursed by insurance.

Another category of agent commonly utilized, as a preventative measure in treating cardiovascular disease are the beta-adrenergic blocking agents. Examples of such agents listed in the current Physicians Desk Reference (PDR 2000) include propanolol, atenolol, timolol maleate, carteolol, penbutolol, nadolol, acebutolol hydrochloride, and metaprolol succinate. Indications for these agents include treatments for hypertension, angina pectoris due to coronary atherosclerosis, cardiac arrythmias, and reduction of cardiovascular mortality in patients who have survived the acute phase of myocardial infarction.

More than 500,000 Americans die from heart disease each year, the leading cause of death in the U.S. The American Heart Association estimates that the total annual cost of medical care and lost productivity due to heart disease is $12 billion to $24 billion. Annually, 1.5 million Americans suffer a heart attack, and people who have had a heart attack are at high risk of having another one. Large studies indicate that tens of thousands of lives could be saved each year if more people were utilizing a beta-blocker after having a heart attack. One study done at the University of Maryland reviewed medical records of more than 200,000 people who had suffered a heart attack, 34% of whom received beta-blockers. During the next two years, people treated with beta-blockers had a 40% lower mortality rate compared to those who had not. Another notable report from Yale University disclosed that one year after a heart attack, patients over 65 years of age who had not taken beta blockers were 14% less likely to be alive than those who had taken them.

The concomitant use of aspirin is generally also indicated in the conditions previously described. It is particularly important in individuals suffering angina pectoris due to coronary atherosclerosis and in individuals who have survived the acute phase of myocardial infarction. Individuals with these disorders are known to commonly utilize many medications. Decreased compliance is known to occur when multiple medications are used.

The problems of achieving compliance with these cardioprotective agents include the inconvenience of taking multiple dosage units over a long period of time, the lack of immediately noticeable beneficial effects from such medications which might otherwise encourage use, trivialization of common medications such as aspirin, inconvenience of the requirement to obtain some medications by prescription and some over-the-counter, unwillingness to make out of pocket purchases, and confusion in older individuals, the age group in which these medications are typically required. Cost factors as well as outcomes must also be considered. Any improvements in compliance can save cost as well as improve outcome.

Simplification is a desired goal. Many of the above-mentioned problems can be ameliorated by incorporating the desired beta-adrenergic blocking agents and antagonists of platelet function into a single dosage unit. Successful prophylactic therapy is clearly preferable and less costly, compared to treatment for symptomatic disease, prolonged illness, and/or disability, which require expensive medical resources including clinic visits, hospitalizations, and major cardiovascular surgery.

Therefore, what is needed is a device and method that combines agents that antagonize beta-adrenergic function and agents that reduce platelet aggregation. What is further needed is a device and method that includes the administration of a single dose.

### SUMMARY OF THE INVENTION

It is an object of the present invention to provide a unitary oral cardiovascular protective medicinal formulation comprising a platelet aggregation inhibitor and a beta-adrenergic antagonist. It is another object of the present invention to provide a method for enhancing compliance with measures for preventing cardiovascular disease by providing an oral formulation comprising a platelet aggregation inhibitor and a beta-adrenergic antagonist, and administering the formulation to a patient in need thereof.

The clear need for cardiovascular preventive treatment, and the failure of patients to avail themselves of such treatment underscores the present need for the formulations of the present invention.

The present invention achieves these and other objectives by providing a system for the treatment of cardiovascular disease that requires a combined single dosage unit regimen and a method for reducing medication error and enhancing therapeutic compliance of combined medication agents for treatment of such disease. The system includes a single dosage unit that combines at least an agent for antagonizing beta-adrenergic function and an agent for reducing platelet aggregation, and preferably instructions for administering the single dosage unit. The single dosage unit may also contain one or more of folic acid, vitamin B6, vitamin B12, and vitamin E. The present invention also includes a method of reducing medication error and enhancing therapeutic compliance of combined agents for the treatment of cardiovascular disease. The method includes formulating in a single dosage unit a beta-adrenergic blocking agent and a platelet inhibitor, and preferably instructing the use of the single dosage unit for treating cardiovascular disease. The method also includes formulating in a single dosage unit one or more of folic acid, vitamin B6, vitamin B12, and vitamin E.

### DETAILED DESCRIPTION OF THE PREFERRED EMBODIMENT

The following detailed description of the invention is provided to aid those skilled in the art in practicing the present invention, however, it should not be construed to unduly limit the present invention. Variations and modifications in the disclosed embodiments may be made by those of ordinary skill in the art without departing from the scope of the present invention.

Compliance with medication is an important consideration in preventing or otherwise treating medical disorders. The simpler the medication regimen, the better the adherence over time. The present invention simplifies the dosing of a plurality of medications for both primary as well as secondary prevention of cardiovascular disease by a single dosage formulation. The present invention simplifies dosing of a plurality of medications for both primary as well as secondary prevention of cardiovascular disease preferably using a single dosage, once-a-day formulation. The present invention provides the components of a regimen for preventing cardiovascular disease in a convenient manner, compared to the current need to purchase individual components.

The present invention provides a single dosage unit that incorporates a beta-adrenergic antagonist and an agent to prevent platelet aggregation in accord with scientific evidence of their efficacy. Other agents may also be incorporated. Examples of other desirable components include the vitamins B6, B12 and folic acid, essential nutritional cofactors in the metabolism of homocysteine. Homocysteine elevation is an independent risk factor in vascular disease and a five-year prospective study has shown that the risk of heart attack for individuals with elevated homocysteine levels is 3.4 times greater in subjects with elevated homocysteine levels. In individuals with elevated homocysteine, lowering of levels usually responds to supplementation with folic acid. In some instances supplementation with vitamins B6 and B12 may also be necessary to lower homocysteine levels.

The inclusion of folic acid in formulations of the present invention in the range of about 200 mcg to about 2000 mcg is considered desirable. It is also desirable to include folic acid, along with B6 in the range of about 2 mg to about 300 mg, or B12 in the range of about 10 mcg to about 1000 mcg, or both, so as to assure normal homocysteine levels.

The naturally occurring antioxidant, vitamin E is another example of an agent that is known to prevent coronary artery disease and strokes and which is considered desirable for inclusion in formulations of the present invention. Epidemiological data has shown a reduction of cardiovascular risk with vitamin E supplementation of at least 100 IU/day. This benefit does not occur at lesser dosages such as a 30 IU/day replacement dosage typical of multivitamin use. In a study of 39,000 health professionals followed for four years, men with a median intake of 419 IU/day of vitamin E had a 44% relative risk reduction compared to men whose median intake was 6 IU/day.

The present invention anticipates that any or all of the active components of the dosage unit may be prepared for immediate release, or if desired, delayed release so as to alter rate of absorption. Materials and methods by which this may be accomplished are well known in the art, for example, by employing hydrophilic matrix materials such as methylcellulose, hydroxyethylcellulose, and hydroxypropylcellulose.

The present invention further anticipates formulations that require dosing schedules of more than once a day, although once-a-day dosing is preferred. The present invention also anticipates that formulations may be in tablet, capsule, caplet, syrup, liquid, or other dosage forms commonly employed for oral administration of medicaments.

The following are examples of proposed formulations of the present invention containing both a beta-adrenergic antagonist and an antiplatelet agent.

### EXAMPLE 1

The synthetic beta1-selective adrenoreceptor blocking agent, atenolol, in a range from about 10 mg to about 100 mg combined with the antiplatelet agent, aspirin, in a range from about 30 mg to about 600 mg to form a single dosage unit. A preferred formulation is a single dosage unit of 25 mg of atenolol combined with 80 mg of aspirin, preferably taken once a day.

### EXAMPLE 2

The formulation of Example 1 which further includes folic acid in a range of about 200 mcg to about 2000 mcg, vitamin B12 in a range of about 10 mcg to about 1000 mcg, vitamin B 6 in a range of about 2 mg to about 300 mg, and vitamin E in a range of about 100 IU to about 800 IU.

### EXAMPLE 3

The synthetic adrenoreceptor antagonist propanalol hydrochloride in a range from about 10 mg to about 300 mg combined with the antiplatelet agent, aspirin, in a range from about 30 mg to about 600 mg to form a single dosage unit. A preferred formulation is a single dosage unit containing 60 mg of propanalol hydrochloride combined with 30 mg of aspirin. The formulation is to be taken three times a day.

### EXAMPLE 4

160 mg of propanalol hydrochloride in a sustained release formulation suitable for once-per-day dosing combined with 80 mg of aspirin. The formulation is to be taken once a day.

### EXAMPLE 5

10 mg of the non-selective beta-adrenoreceptor blocking agent timolol maleate combined with 30 mg of aspirin. This formulation might be taken twice a day for long-term prophylactic use in patients who have survived the acute phase of myocardial infarction.

### EXAMPLE 6

100 mg of the beta1-selective beta-adrenoreceptor blocking agent metoprolol tartrate combined with 80 mg of aspirin. This formulation might be taken twice a day for long-term prophylactic use in patients who have survived the acute phase of myocardial infarction.

These examples are not meant to be inclusive and it is contemplated that other dosages, other beta-blocking agents, and other platelet-active agents that exert preventative effects on cardiovascular disease by altering platelet adhesion, aggregation, and/or release of platelet factors, when incorporated together into a single formulation, are within the scope of this invention.

Various modifications and alterations of the present invention may be appreciated based on a review of this disclosure, and such changes and additions are intended to be within the scope and spirit of this invention as defined by the following claims.

What is claimed is:

1. A medicament dosage unit consisting essentially of a beta-adrenergic blocker and a platelet inhibitor together in a single dosage unit quantity sufficient to provide a cardiovascular protective dosage.

2. The dosage unit of claim 1 wherein said dosage unit is formulated as a once-a-day dosage unit.

3. The dosage unit of claim 1 wherein said platelet inhibitor is aspirin.

4. The dosage unit of claim 1 wherein said beta-adrenergic blocker is atenolol.

5. The dosage unit of claim 1 wherein said beta-adrenergic blocker is propanolol.

6. The dosage unit of claim 1 wherein said beta-adrenergic blocker is timolol.

7. The dosage unit of claim 1 wherein said beta-adrenergic blocker is metaprolol.

8. A method of treating cardiovascular disease said method comprising:
formulating a single dosage unit consisting essentially of a beta-adrenergic blocking agent and a platelet inhibitor in a single dosage unit quantity sufficient to provide a cardiovascular protective dosage; and
administering said single dosage unit to a patient.

9. The method of claim 8 further comprising indicating the use of said dosage unit for treating cardiovascular disease.

10. The method of claim 8 further comprising providing instructions for administering said single dosage unit.

11. The method of claim 8 wherein said formulating step further includes formulating said single dosage unit as a once-a-day dosage unit.

12. A method of making a cardiovascular protective dosage unit comprising formulating a single dosage unit consisting essentially of a beta-adrenergic blocking agent and a platelet inhibitor in a quantity sufficient to provide a cardiovascular protective dosage.

13. The method of claim 12 wherein said platelet inhibitor is aspirin.

14. A medicament dosage unit consisting essentially of a beta-adrenergic blocker and aspirin combined in a single dosage unit in sufficient quantity to provide a cardiovascular protective dosage.
